(19) United States
(12) Patent Application Publication
(10) Pub. No.: US 2021/0259947 A1
(43) Pub. Date: Aug. 26, 2021

(54) METHOD FOR OBTAINING AN AQUEOUS EXTRACT OF DUNALIELLA SALINA AND COSMETIC USES OF SAME

(71) Applicants: ISP INVESTMENTS LLC, Wilmington, DE (US); SEACRET SPA LTD, Tel Aviv (IL)

(72) Inventors: Elodie OGER, Vallauris (FR); Isabelle IMBERT, Cannes (FR); Audrey LE MESTR, Antibes (FR); Rachel CHABERT, Grasse (FR); Isabelle AFRIAT STALOFF, Maale Adumin (IL); Boaz YADIN, Tel Aviv (IL)

(73) Assignees: ISP INVESTMENTS LLC, Wilmington, DE (US); SEACRET SPA LTD, Tel Aviv (IL)

(21) Appl. No.: 16/973,878

(22) PCT Filed: Jun. 14, 2019
(86) PCT No.: PCT/EP2019/065655

§ 371 Date: Dec. 10, 2020

(30) Foreign Application Priority Data
Jun. 15, 2018 (FR) 1870710

(51) Int. Cl. A61K 8/9722; C12N 1/12; A61K 8/9728; A61K 8/60; A61Q 5/12

(52) U.S. Cl. CPC A61K 8/9722; C12N 1/12; A61K 8/9728; C12N 2500/74; A61Q 5/12; C12N 2500/34; C12N 2500/12; A61K 8/60

(57) ABSTRACT

Methods for obtaining an aqueous extract of a microalgae Dunaliella salina cultivated in the absence of light in a culture medium of a yeast extract, sugar and salt include solubilising a yeast extract in water then adding sugar and salt. After solubilisation of the salt and at a pH between 5 and 8, the microalgae Dunaliella salina is added with stirring, in darkness and at room temperature, for a fermentation period of at least 12 hours. After the fermentation period, grinding and filtering are performed to separate a soluble aqueous raw extract to which salt is added. This extract undergoes sterilising filtration with a porosity threshold of less than or equal to 0.2 μm and has a pH between 3.5 and 4.5. The extract obtained by the method, compositions incorporating the extract, and methods of skincare, scalp care and skin appendage care using such compositions are also disclosed.

### TECHNICAL FIELD

The present invention relates to the field of cosmetics. It relates more particularly to a method for obtaining an aqueous extract of a Dunaliella salina microalgae, the extracts likely to be obtained by such a method and cosmetic compositions comprising such extracts, as well as their cosmetic uses for skincare, scalp care and skin appendage care.

The skin is a vital organ composed of a plurality of layers (dermis, proliferative layers and stratum corneum), which cover the entire surface of the body and perform protective, sensitive, immune, metabolic or thermoregulatory functions. The skin is the interface between the body and the external environment. It aims to protect the body from external aggressions but also to fight against dehydration by limiting the diffusion of water. This cutaneous barrier is largely provided by the epidermis. The skin, like other organs, is subject to aging.

The appearance of the skin can be modified by alterations that are internal (intrinsic aging, diseases and hormonal changes such as pregnancy) or external (environmental factors such as pollution, sunlight, pathogens, temperature variations, etc.). Following these alterations, wrinkles and fine lines, pigmentation defects, dryness or even dehydration of the skin, thinning of the epidermis, elastosis, blemishes, age spots, etc. may appear. All these changes affect not only the skin, but also the keratinous appendages such as nails and hair.

The term microalgae (or microphyte), on the other hand, refers to microscopic algae. Dunaliella salina is a type of green, unicellular, bi-flagellar and halophilic microalgae belonging to the family Chlorophyceae, which includes about 30 genera including Dunaliella. There are about ten species of the genus Dunaliella, the best known and studied being D. tertiolecta, D. media, D. euchlora, D. minuta, D. parva, and D. viridis. It should be noted that not all of the above-mentioned species tolerate salt concentrations as high as D. salina. Some Dunaliella species are marine organisms that have never been reported in hypersaline environments.

As a result, few organisms can survive, like Dunaliella salina, in conditions with such high salt concentrations. Dunaliella species are indeed able to tolerate varying concentrations of NaCl ranging from 0.2% to about 35%. Also, in the Dead Sea, Dunaliella species are mainly found, especially Dunaliella salina (Volacani B E, 1944). The Dead Sea lies along the Syrian-African fault line. It is the result of a geological phenomenon that began three million years ago. During a gigantic natural upheaval, several layers of mineral-rich soil came to the surface, and springs gushed out, giving rise to a valley and a lake below sea level. Evaporation that continued for millennia resulted in the concentration of salts and minerals in the waters of the lake that is now called the Dead Sea to a level not found in any other sea or ocean. Thus, the waters of the Dead Sea contain 3 times more sodium, 30 times more magnesium, 16 times more potassium and 36 times more calcium than the Mediterranean Sea, for example. The microalgae Dunaliella salina is one of the few species that can live in an environment in which the salinity is so significant. Dunaliella salina also has a very high pH tolerance ranging from pH 1 to pH 11 and is also capable of withstanding temperatures below 0° C. and above 38° C.

Since Dunaliella cells do not have a rigid cell wall, they produce a large amount of glycerol to cope with high salt concentration and provide protection against osmotic pressure. Thus, glycerol acts as a “compatible solute” that specifically protects enzymes from inactivation and inhibition (Brown and Borowitzka, 1979). In addition, to survive exposure to sometimes intense light, these microalgae synthesise carotenoids. This large amount of carotenoids also provides antioxidant activity. Dunaliella salina was first noticed in salt evaporation basins in the south of France in 1838 by Michael Felix Dunal and named after its discoverer by Teodoresco in 1905. After this discovery, Dunaliella salina became a model organism for the study of salt adaptation. The establishment of the concept of solutes compatible with organic compounds to achieve osmotic equilibrium is indeed largely based on the study of Dunaliella species.

The shape of the cell in Dunaliella species varies from ellipsoid, ovoid, cylindrical, pyriform and fusiform to almost spherical. Cells of a given species can change shape depending on environmental conditions, often becoming spherical under adverse conditions.

Cell size may also vary to some extent with growing conditions and light intensity (Marano, 1976; Riisgård, 1981; Einsphar et al., 1988).

Microalgae have been consumed for thousands of years around the world. For example, traces of the consumption of various species of microalgae in Mexico during the time of the Aztecs have been found. Europe and industrialised countries use microalgae as food supplements to combat malnutrition, as well as for aquaculture. These microalgae, in particular Dunaliella salina, are notably used for alternative fuel solutions, called biofuel, which represent the primary use of this microalgae in the world. In these cases, they are cultivated outdoors in “raceway” ponds or in closed environments, in photobioreactors. Indeed, Dunaliella species, and in particular Dunaliella salina, are known for their ability to produce and accumulate large amounts of lipids, β-carotene in droplet form, so that the cells appear red-orange rather than green. These molecules produced by the Dunaliella family have applications in fields as varied as biofuel production, cosmetic or nutraceutical.

### PRIOR ART

Today, the most common way to cultivate microalgae, and in particular Dunaliella salina, is cultivation under autotrophic conditions in open ponds. In this type of culture, microalgae capture light energy and use CO2 as a carbon source, performing photosynthesis. It is then sufficient for the culture medium to contain mineral elements for the microalgae to multiply. However, photo-autotrophic culture has a low yield. Biomass production is indeed severely limited due to self-shading caused by the increase in biomass during cultivation, which prevents the availability of light towards the end of growth. In addition, this type of cultivation requires a high cost.

Some microalgae such as Dunaliella can also grow in mixotrophic conditions. In this case, they use both an organic carbon source and light energy.

Finally, Dunaliella can also grow under so-called heterotrophic culture conditions: the microalgae are cultivated in the dark, in closed or open bioreactors. They must then have a source of organic carbon, such as ethanol, acetate and glucose.

All these cultivation methods are widely described in the scientific literature (Bumbak et al., 2011).

The advantage of bioreactor production is that it can be well controlled. It is indeed possible to control all the culture parameters, pH, temperature, culture medium, sugar levels or other molecules necessary for the growth of microalgae. The disadvantage is that bioreactor culture represents a cost to acquire all the equipment required for this type of culture.

In general, the culture medium under the heterotrophic condition of microalgae such as Dunaliella must contain molecules that are essential for their growth in the absence of light. The culture media used are often synthetic culture media containing salt, macro- and micro-nutrients, vitamins, mineral salts, as found in Johnson's modified medium (Johnson et al., 1968; Borowitzka, 1988) widely used for the culture of Dunaliella. These environments must ensure the growth of Dunaliella by providing carbohydrates. Dunaliella microalgae also need a source of nitrogen for their growth, which can be provided either in the form of nitrate, ammonia, urea or by using a yeast extract frequently used for the culture of bacteria, yeast. Phosphorus is also a major nutrient for the growth of microalgae because of its involvement in several cellular processes such as energy transfer and the synthesis of nucleic acids and phospholipids. This phosphorus is mainly assimilated in inorganic form (H2PO4- or HPO42-) (Barsanti and Gualtieri, 2006).

We know, for example, is the document JP2003325165, which describes a method of cultivating Dunaliella in concentrated seawater for its use in thalassotherapy. Dunaliella can be used in powder or paste form.

We also know the publication by Gladue et al. (“Microalgal feeds for aquaculture, Journal of applied phycol, Kluwer, Dordrecht, N L, vol. 6 no. 2, 1 Apr. 1994, pages 131-14), which discloses a microalgae culture medium comprising seawater (80%), glucose (1.8%) and yeast extract (0.05%). D. salina is grown in this culture medium in the absence of light with moderate agitation for a period of at least 24 hours with a pH of 7.5-8.

We also know the document WO2013058431, which describes a method for growing Dunaliella in natural seawater with the addition of charcoal and NaOH.

Patent documents are known (e.g. CN102851214, CN102936569, CN104988065, KR2006000307, or CN1923994), describing different ways of growing Dunaliella salina in media including a nitrogen source (KNO3, NH4NO, CO(NH2)2, (NH4)2CO3, NH4NO3, NaNO3), a source of phosphorus and/or potassium (KH2PO4, KNO3, Na3PO4), a source of inorganic carbon (NaHCO₃), salt (NaCl or sea water) and optionally vitamins or ionic compounds for enhancing the growth of Dunaliella salina and the production of beta-carotene.

In particular, patent document CN1446904 is known which describes the cultivation of Dunaliella salina for 2 to 3 weeks from Dunaliella salina powder in salt water, in light, at a temperature of between 10 and 35° C., the culture medium having a medium pH of between 6 and 8 and comprising NaH2PO4 and CH4N2O. This alga can be used for cosmetic or therapeutic care and for genetic engineering purposes.

Dunaliella salina is thus known to be used in particular in cosmetics partly for the use of the β-carotenes it contains, known for their antioxidant activity and essential for the synthesis of vitamin A. The β-carotene protects the skin from the harmful rays of the sun and is transformed into vitamin A in the skin, which allows its renewal. Dunaliella salina also contains many amino acids (including glycine and alanine) and essential fatty acids enriched with vitamins E and B. These elements are fundamental in the fight against skin aging. Dunaliella salina also contains a high amount of glycerol which has important water absorption and retention properties.

Moreover, despite the many anti-aging cosmetic products on the market for skin treatment, so-called “unnatural” and chemically synthesised products can be perceived as dangerous for the environment as well as for people. Natural products, on the other hand, are generally better perceived.

Although many natural products extracted from plants or algae are known to contain phytocompounds that can have beneficial effects on the skin, there is still a need for new, effective cosmetic compositions applied topically that have anti-aging, moisturising effects for skin and hair, using natural ingredients as the active agent.

The extract of Dunaliella salina described in the present invention has the advantage of being obtained from a culture of Dunaliella salina carried out under heterotrophic conditions, in the presence of a completely natural culture medium, unlike other extracts already known on the market and using synthetic culture media. The benefit of heterotrophic culture allows the culture of Dunaliella salina to be carried out in the absence of light, which allows a mode of culture more easily adaptable on a large scale without the need for apparatus specifically dedicated to the culture of microorganisms such as microalgae.

### Technical Problem

In view of the foregoing, a problem which the present invention proposes to solve is to provide a new method of cultivating Dunaliella salina to obtain an extract enriched with compounds of benefit for skin care. Such natural extracts according to the invention thus have improved anti-aging and moisturising effects.

### Technical Solution

The solution to this problem is firstly a method for obtaining an aqueous extract of a microalgae Dunaliella salina, in which said microalgae is cultivated in the absence of light in a culture medium comprising a yeast extract, sugar and salt according to the following steps:

a) solubilising a yeast extract in water;
b) adding glucose to the mixture obtained in a);
c) adding Dead Sea salt;
d) after total solubilisation of the salt in the mixture obtained in b), the culture medium thus obtained has a pH of between 5 and 8, and the microalgae Dunaliella salina is added thereto;
e) moderately stirring the mixture obtained in d), in darkness and at room temperature, for a period of at least 12 hours, in order to allow the fermentation of the microalgae Dunaliella salina;
f) grinding and then filtering the fermented Dunaliella salina mixture obtained in e) in order to separate the soluble and insoluble materials;
g) recovering a soluble aqueous raw extract to which Dead Sea salt or a preservative such as sodium benzoate is added;
h) carrying out sterilising filtration with a porosity threshold of less than or equal to 0.2 μm; and
i) obtaining a fermented aqueous extract of Dunaliella salina in which the pH is between 3.5 and 4.5.

A second object is an aqueous extract of Dunaliella salina likely to be obtained by the method according to the invention, comprising, by weight of the total weight of the extract, from 15 to 25 g/kg dry weight, 0.1 to 2 g/kg protein fragments, 0.3 to 3 g/kg sugars, 0.5 to 3 g/kg amino acids, and 20 to 150 mg/kg phenolic compounds.

The third object is a composition comprising, as an active anti-aging agent, an effective amount of an aqueous extract of Dunaliella salina according to the invention, and a physiologically acceptable medium.

Finally, the last object of the invention is the cosmetic use of a composition according to the invention for skincare, scalp care and skin appendage care.

### Advantages Provided

The inventors have developed a new method of growing Dunaliella salina based on the heterotrophic culture system, in which Dunaliella salina produces compounds of benefit by fermentation.

One of the advantages of the present invention is that the microalgae Dunaliella salina is used alive, cultivated under heterotrophic conditions in a specific and completely natural culture medium consisting of a yeast extract, sugar and salt from the Dead Sea. All the elements used to grow the microalgae are natural, with no synthetic molecules added to the culture medium. Fermentation will take place because the nutrient medium contains all the compounds necessary for the growth of the microalgae. The various compounds will be metabolised by the microalgae. Dunaliella salina, to allow it to grow. Indeed, the use of a yeast extract provides the nitrogen sources necessary for the growth of the microalgae, and the addition of glucose provides the carbon source also necessary for the growth of the microalgae. Moreover, the fact that Dunaliella salina has to operate in a saline environment is made possible by the addition of Dead Sea salt to provide all the essential minerals. The benefit of using Dead Sea salt also comes from its beneficial properties on the skin, which have long been recognised in cosmetics. Its waters, very rich in oligo-minerals, bestow soothing and beneficial properties on the skin. Indeed, the composition of the waters of the Dead Sea is unique and contains higher concentrations of magnesium, potassium, silica, sodium and calcium than any other salt water in the world, including the oceans.

The fermentation of Dunaliella salina microalgae under these culture conditions according to the invention makes it possible to obtain, after extraction of the cell biomass, a final extract highly enriched in various compounds of benefit from the microalgae but also from its culture medium. The latter can be preserved during extraction, as it is composed exclusively of natural molecules. This makes it possible to offer a new extract from Dunaliella salina, different from an extract made only from dried Dunaliella salina.

The extract of Dunaliella salina fermented according to the invention is rich in compounds of benefit such as amino acids, monosaccharides, proteins and peptides. Associated with the mineral salts of Dead Sea salt, all of these water-soluble molecules, known for their beneficial effects on the skin, contribute to the improved efficacy of the extract according to the invention compared to an unfermented extract of Dunaliella salina.

In this description, unless otherwise specified, it is understood that, when an interval is given, it includes the upper and lower limits of that interval.

### BRIEF DESCRIPTION OF THE DRAWINGS

The invention and the advantages deriving therefrom will be better understood by reading the following description and the non-limiting modes of implementation that follow, written in relation to the appended figures in which:

FIG. 1 shows the growth of Dunaliella salina in culture according to the method of Example 1 according to the invention.

FIG. 2 shows the consumption of hexose of Dunaliella salina in culture according to the method of Example 1 according to the invention.

FIG. 3 shows the effect of the presence of Dunaliella salina on the concentration of amino acids (proteins) contained in a culture medium according to the method of Example 1 according to the invention.

FIG. 4 shows the evaluation of an aqueous extract of fermented Dunaliella salina obtained by the method of Example 1 according to the invention on the barrier function and movement of water on skin ex vivo.

### DESCRIPTION OF EMBODIMENTS

The invention relates to a method for obtaining an aqueous extract of fermented Dunaliella salina from live Dunaliella salina, cultivated under heterotrophic culture conditions, in a medium comprising a yeast extract, sugar and salt, and in the absence of light. Cultivation is advantageously carried out in an open environment.

Dunaliella is understood to mean all microalgae of the species Dunaliella. Dunaliella is a microalgae capable of growing in different types of environment: halotrophic (energy from light), heterotrophic (energy from carbon input), mixotrophic (mixture of light and carbon).

Preferably, the extract is obtained from the fermentation of Dunaliella salina.

Fermentation is understood to mean the cultivation of the microalgae in a specific nutrient medium according to the invention, containing in particular a source of organic carbon, which the microalgae will metabolise in order to grow.

More specifically, the fermentation of Dunaliella salina microalgae under heterotrophic conditions means the culture in a medium containing all the nutrients necessary for the growth of the microalgae and where the contribution of carbon sources, especially glucose, will allow the multiplication of the microalgae in an environment devoid of light (Barclay et al. 1994, Heterotrophic production of long chain omega-3 fatty acids utilizing algae and algae-like microorganisms; Garcia et al. 2013, A method for biodiesel production involving the heterotrophic fermentation of Chlorella protothecoides with glycerol as the carbon source).

The aqueous extract of the microalgae Dunaliella salina according to the invention is obtained from the method comprising the following steps:

a) solubilising a yeast extract in water;
b) adding glucose to the mixture obtained in a);
c) adding Dead Sea salt;
d) after total solubilisation of the salt in the mixture obtained in b), the culture medium thus obtained has a pH of between 5 and 8 to, and the microalgae Dunaliella salina is added thereto;
e) moderately stirring the mixture obtained in d), in darkness and at room temperature, for a period of at least 12 hours, in order to allow the fermentation of the microalgae Dunaliella salina;
f) grinding and then filtering the fermented Dunaliella salina mixture obtained in e) in order to separate the soluble and insoluble materials;
g) recovering a soluble aqueous raw extract to which Dead Sea salt or a preservative such as sodium benzoate is added;
h) carrying out sterilising filtration with a porosity threshold of less than or equal to 0.2 μm; and
i) obtaining a fermented aqueous extract of Dunaliella salina in which the pH is between 3.5 and 4.5.

Dunaliella salina is one of the few living species capable of living in the Dead Sea. Indeed, while the average salinity of seawater varies between 2 and 4%, that of the Dead Sea is approximately 27.5% (or 275 grams per litre).

The Dead Sea is a saltwater lake. Its water is very rich in minerals and trace elements and has many properties. Natural salt from the Dead Sea has twenty-six vital minerals, including calcium, potassium and magnesium. The natural salt of the Dead Sea, contains more particularly on average: potassium (120,000 mg/I), magnesium (85,000 mg/I), chlorine (38,000 mg/I), sodium (23,000 mg/I), calcium (22,000 mg/I), bromine (5600 mg/I), carbonate, chromium, phosphorus, iron, etc. Dead Sea salt, because of its richness in various essential minerals, is widely used in cosmetics, and recognised in particular for its beneficial properties on the skin. It has a disinfectant and repairing action on the skin (Proksch et al., Int J Dermatol. 2005; 44(2):151-7).

The calcium it contains promotes cellular exchanges.

Magnesium contributes to slowing down the aging process and helps to relax the muscles. It also relieves the joints. It is considered a natural anti-stress.

Potassium helps to balance skin hydration. It therefore helps to restore the water balance.

In a first step a) of the method according to the invention, a yeast extract, for example in powder form, is dissolved with water, preferably in a ratio of yeast extract/water of 0.1 to 2% w/w, more preferably in a ratio of 0.2%.

The water used is distilled, demineralised water or water rich in mineral salts and/or trace elements, preferably distilled water.

Preferably, the yeast extract is an extract of Saccharomyces cereviseae.

Yeast extract can be in the form of powder or fresh yeast extract. Preferably, the yeast extract is in powder form.

Glucose is then added in step b) to the mixture obtained in a).

The glucose concentration is preferably between 0.1 and 4%, and more preferably 0.2%. This concentration is chosen to optimise the fermentation yield of the microalgae Dunaliella salina.

In step c), once the glucose and the yeast extract are solubilised, Dead Sea salt is added, preferably between 1 and 5%, more preferably 2.6%, which will provide the microalgae with all the mineral salts essential for its development, and to obtain a salinity of water essential for the development of the microalgae, the pH of the mixture should be between 5 and 8;

For example, the pH is adjusted by adding a solution of hydrochloric acid (HCl) or any pH-regulating acid that is compatible with cosmetic use such as citric or lactic acid.

This mixture will constitute an optimal culture medium to host the microalgae and provide it with all the nutrients necessary for its development, growth and cell division.

In step d), the microalgae Dunaliella salina is added to this soluble nutrient solution with a pH between 5 and 8.

According to an advantageous embodiment of the method according to the invention, between 0.5% and 10% of Dunaliella salina inoculum is preferably added to the culture medium, more preferably between 1% and 5%, even more preferably 2%.

In step e), the mixture obtained in d) is kept under moderate agitation, in darkness and at room temperature, for a period of at least 12 hours, to allow the fermentation of the Dunaliella salina microalgae.

Fermentation is carried out when Dunaliella salina is cultured in such a specific nutrient medium according to the invention containing a yeast extract, glucose and Dead Sea salt, in water at room temperature or with water heated to a maximum temperature of 35° C. for at least 12 hours, advantageously in an open medium.

In step f), the fermented Dunaliella salina mixture obtained in e) is grinded and then filtered to separate the soluble and insoluble materials so as to recover in step g) a soluble aqueous raw extract to which Dead Sea salt or a preservative such as sodium benzoate is added.

The addition of Dead Sea Salt acts as a preservative.

In step h), a sterilising filtration with a porosity threshold less than or equal to 0.2 μm is carried out.

Finally in step i), a fermented aqueous extract of Dunaliella salina is obtained in which the pH is between 3.5 and 4.5, preferably between 3.5 and 4.0, more preferably the pH is 4.0.

Preferably, in steps d) and i), the pH is controlled and possibly readjusted by adding a solution of hydrochloric acid (HCl) or sodium hydroxide (NaOH).

The aqueous extract of Dunaliella salina likely to be obtained by the method according to the invention is composed in particular of amino acids, sugars, protein fragments and phenolic compounds.

The extract thus obtained according to the invention is a clear and brilliant solution.

Preferably, such an aqueous extract of Dunaliella salina likely to be obtained by the method according to the invention comprises, by weight of the total weight of the extract, from 15 to 25 g/kg dry weight, 0.1 to 2 g/kg protein fragments, 0.3 to 3 g/kg sugars, 0.5 to 3 g/kg amino acids and 20 to 150 mg/kg phenolic compounds.

Preferably, an aqueous extract of Dunaliella salina likely to be obtained by the method according to the invention comprises, by weight of the total weight of the extract, 0.5-1 g/kg of protein fragments, 1-2.5 g/kg of sugars, 0.6-2 g/kg of amino acids and 40-100 mg/kg of phenolic compounds.

The aqueous extract according to the invention does not comprise a detectable amount of lipophilic molecules such as carotenoids.

Another object of the invention relates to a composition comprising, as an active anti-aging agent, an effective amount of an aqueous extract of Dunaliella salina likely to be obtained by the method according to the invention, and a physiologically acceptable medium.

An effective quantity means the minimum quantity of extract according to the invention which is necessary to obtain the activity of the extract, in particular cosmetic and more particularly to improve the appearance of the skin, to fight against the signs of skin aging or to improve the hydration of the skin, without this quantity being toxic.

A physiologically acceptable medium means a vehicle suitable for contacting the outer layers of the skin or mucous membranes, without toxicity, irritation, undue allergic and similar response, or intolerance reaction, and proportionate to a reasonable benefit/risk ratio.

Examples of physiologically acceptable media commonly used in the intended field of application may include adjuvants necessary for formulation such as solvents, thickeners, diluents, antioxidants, dyes, sunscreens, self-tanning agents, pigments, fillers, preservatives, fragrances, odour absorbers, essential oils, vitamins, essential fatty acids, surfactants, film-forming polymers, etc.

Preferably, the composition according to the invention comprises the aqueous extract of Dunaliella salina likely to be obtained by the method according to the invention at a concentration of 0.1 to 10% by weight of the total weight of the composition, preferably 0.5% to 5%.

The composition usable according to the invention may be applied by any appropriate means, in particular topically externally, and the formulation of the compositions shall be adapted by a person skilled in the art.

Preferably, the compositions according to the invention are in a form suitable for topical application. These compositions must therefore contain a physiologically acceptable medium, i.e. compatible with the skin and skin appendage, without risk of discomfort during application, and cover all suitable cosmetic forms.

Topical application means applying or spreading the aqueous extract of Dunaliella salina likely to be obtained by the method according to the invention, and more particularly a composition containing it, to the surface of the skin, mucous membrane or skin appendage.

“Skin” means the skin of the face, including the eye contour and mouth, nose, forehead, neck, hands, but also the skin of the entire body.

Skin appendage means substances naturally present in the human or animal body that are rich in keratin, especially hair, eyelashes, eyebrows and nails.

The topical compositions for implementing the invention may in particular be in the form of an aqueous, hydro-alcoholic or oily solution, an oil-in-water or water-in-oil emulsion, multiple emulsion, micro-emulsion, nanoemulsion or any colloidal system usable in cosmetics; they may also be in the form of suspensions, or even powders, suitable for application to the skin, mucous membranes, lips and/or hair.

These compositions can be more or less fluid and also have the appearance of a cream, lotion, milk, serum, ointment, gel, paste or foam. They can also be in solid form, such as a stick, or applied to the skin as an aerosol.

In all cases, the person skilled in the art shall ensure that the adjuvants and their proportions are chosen in such a way so as not to prejudice the desired advantageous properties of the composition according to the invention. These adjuvants may, for example, correspond to 0.01 to 20% of the total weight of the composition. When the composition according to the invention is an emulsion, the fatty phase may represent from 5 to 80% by weight and preferably from 5 to 50% by weight of the total weight of the composition. The emulsifiers and co-emulsifiers used in the composition are selected from those classically used in the field in question. For example, they can be used in a proportion ranging from 0.3 to 30% by weight of the total weight of the composition.

A final object of the invention relates to the cosmetic use of a composition according to the invention for skincare, scalp care and skin appendage care. The invention relates in particular to the cosmetic use of the composition according to the invention to improve the appearance of the skin, to combat the signs of skin aging or to improve the hydration of the skin and strengthen the barrier function.

The use and compositions according to this invention are particularly intended for skincare and skin appendage care.

The invention relates more particularly to the cosmetic use of a composition according to the invention to improve the hydration of the skin and strengthen the barrier function.

The invention also relates more particularly to the cosmetic use of a composition according to the invention for combating the signs of skin aging and improving the appearance of the skin, in particular the firmness and elasticity of the skin.

By “improving the appearance of the skin”, we mean that the skin grain appears finer, the luminosity more intense and the complexion more homogeneous.

“Signs of cutaneous aging” means any changes in the external appearance of the skin due to aging such as, for example, fine lines and wrinkles, cracks, bags under the eyes, dark circles, wilting, loss of elasticity, firmness and/or tone of the skin, but also any internal skin modifications that do not systematically result in a modified external appearance such as, for example, thinning of the skin, or any internal skin damage resulting from environmental stresses such as pollution and UV radiation.

“Improvement in skin hydration” means any improvement in the changes in the external appearance of the skin due to dehydration such as dryness, tightness and discomfort.

### EXAMPLES

The present invention will now be illustrated by way of the following examples.

### Example 1: Preparation of a Fermented Dunaliella salina Extract According to the Invention

The preparation of the fermented Dunaliella salina extract will take two days. On the first day, the nutrient culture medium in which the microalgae will grow is prepared.

To prepare 1 kg of culture medium, 0.2% yeast extract powder (autolysed extract of Saccharomyces cerevisiae), i.e. 2 g of powder to which approximately 800 ml of distilled water is added, is placed in a beaker. The solution is agitated until the powder is completely dissolved. Once the yeast powder is well solubilised, 0.2% glucose is added, i.e. 2 g of glucose is added to the solution previously obtained. The solution is agitated constantly during the preparation time. Once the sugar is completely solubilised, 2.6% Dead Sea salt is added, i.e. 26 g. The solution is kept under agitation until the salt is completely dissolved. The pH of the nutrient medium is then measured and should be between 5.5 and 8 and if necessary optimally adjusted between 6 and 7. The culture medium is then ready to receive the microalgae Dunaliella salina. 2% Dunaliella salina inoculum is added to the culture medium. Inoculum means a concentrated liquid culture of Dunaliella salina in which the microalgae are alive. Distilled water is added to obtain a final total weight of 1 kg. The pH of the final solution is then measured and adjusted if necessary between 6 and 7, the optimal culture pH for the Dunaliella salina microalgae. The solution is kept under agitation for a minimum of 12 hours in darkness and at room temperature, in an open environment. This culture is known as heterotrophic because the fermentation of Dunaliella salina takes place in darkness in the presence of an organic carbon source. After the night of fermentation of the Dunaliella salina, on the second day, a grinding step is carried out to release the cell content of the microalgae, in order to enrich the culture medium as much as possible. Subsequently, a filtration step with a carbon-containing filter is carried out to deodorise the extract and separate the solid debris from the liquid phase to obtain a clear extract. Subsequently, the purification of the extract is done by a series of filtrations of decreasing porosity ranging from 1 μm to 0.3 μm. The pH of the extract is set between 3.5 and 4.5. Finally, a final stage of sterilising filtration is carried out with a porosity of 0.2 μm. The extract thus obtained is what is referred to in the present invention as the preservative-free version, which was used for the biological evaluation tests described below in Example 5.

Optionally, preservatives can be advantageously added to the extract thus obtained, for example 8% dead sea salt or sodium benzoate.

The extract of Dunaliella salina thus obtained is analysed and has the following characteristics: weight of dry matter: 22 g/kg; total protein content (protein fragments): 0.5 g/kg; total sugar content: 2.3 g/kg; free amino acid content: 0.6 g/kg; and total polyphenol content: 40 mg/kg.

The total protein content (protein fragments) of Dunaliella salina extract was determined by a Lowry protein assay (Lowry et al., 1951). The absorbance of the sample is read on a spectrophotometer at 550 nm. The protein content is determined using a standard BSA curve.

The amino acid content of the extract was determined from a protocol published by Moore (Moore et al., 1948). The free amino acid content of the extract was evaluated by the formation of a coloured complex, following the breakdown of the amine and carboxylic functions by the ninhydrin reagent. The absorbance of the complex is read on a spectrophotometer at 570 nm. The total amino acid content is determined in relation to a pool of amino acids as standard.

The total sugar content of the extract was determined by adapting the assay described by Dubois (Dubois et al., “Colorimetric method for the determination of sugars and related substances”, Anal. Chem., 1956, 28 (3), 350-356). This analysis consists of dissolving the raw material in concentrated sulphuric acid and then reacting with phenol to form a coloured complex. The absorbance of the complex is read on a spectrophotometer at 490 nm. The sugar content is determined using a standard glucose curve.

The polyphenol content of the extract was determined using the Folin-Ciocalteu assay (Singleton et al., Analysis of total phenols and other oxidation substrates and antioxidants using the Folin-Ciocalteu reagent, 1999, 299: 152. Polyphenol compounds in the sample react with the Folin-Ciocalteu reagent, oxidation of the reagent gives a blue colour. The absorbance of the sample is read on a spectrophotometer at 760 nm.

The content was expressed in gallic acid equivalents using a standard gallic acid curve.

### Example 2: Characterisation of the Fermented Dunaliella salina Extract Obtained According to the Invention

In general, an aqueous extract of Dunaliella salina is obtained which is very pale green in colour, limpid, glossy, containing 15 to 25 g/kg dry weight extract, 0.1 to 2 g/kg protein fragments, 0.3 to 3 g/kg sugars, 0.5 to 3 g/kg amino acids, and 20 to 150 mg/kg phenolic compounds.

In the extraction of Example 1, an aqueous extract of 22 g/kg dry weight extract was obtained. This extract is called “fermented non-preserved” extract and has been used for all biological analyses.

Physico-chemical analysis shows that this extract has a concentration of 0.5 g/kg protein fragments, 2.3 g/kg sugars, 640 mg/kg amino acids, and 40 mg/kg phenolic compounds.

Optionally, the extract thus obtained can be advantageously preserved by adding Dead Sea salt at a concentration of 8% or a preservative such as sodium benzoate. The extract is then called “preserved extract”.

### Example 3: Production of an “Unfermented” Extract of Dunaliella salina

To obtain an extract of Dunaliella salina known as “unfermented”, the extract is made by the method as described in Example 1, the only difference being that the solution containing the culture medium and the microalgae is not agitated for 12 hours in the dark, during which time the microalgae ferments. As soon as the microalgae is added to the medium, the solution containing the nutrient medium and the microalgae is immediately grinded and filtered as described in Example 1. This makes it possible to obtain an extract in which the microalgae have not undergone heterotrophic fermentation and thus to carry out comparative analyses from both a physico-chemical and biological assessment point of view.

### Example 4: Demonstration of the Fermentation of Dunaliella salina Under the Growing Conditions of Example 1

In order to demonstrate that the microalgae Dunaliella salina is capable of growing under the cultivation conditions described according to the invention (Example 1), modifications of certain cultivation parameters and analyses of certain biological markers were carried out.

One of the first culture parameters to be measured when monitoring the growth of a microorganism in culture is its biomass over time. Cell density is measured by optical density (OD), which is a reliable parameter for measuring growth. For this purpose, OD was measured by spectrophotometry at a wavelength of 700 nm commonly used to define the opacity of a culture of a microorganism.

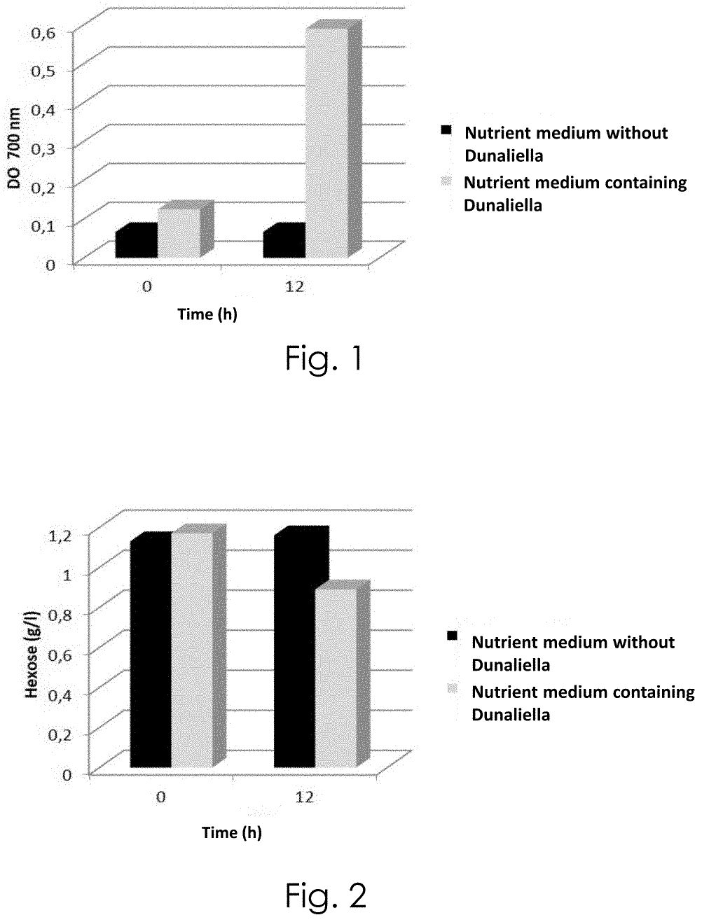

FIG. 1 shows the OD measured in darkness and at room temperature at t=0 and t=12 h of the culture medium alone (or nutrient medium), i.e. without the addition of the microalgae Dunaliella salina, and the culture medium with the addition of the microalgae Dunaliella salina. A significant increase in OD is observed after 12 hours in the presence of Dunaliella salina. This increase is due to the growth of the Dunaliella salina which fermented and divided during this 12 hour-period.

It is also possible to demonstrate the growth of microorganisms in culture by their consumption of substances necessary for their growth. This is the case of sugar, which is an essential nutrient for the cell division of microorganisms. Indeed, the consumption of the carbon source of the culture medium, for example glucose (hexose), is a recognised culture monitoring parameter for monitoring the growth of microorganisms. A comparative analysis of the concentration of hexose in culture medium with and without the microalgae Dunaliella salina at room temperature at t=0 and t=1 2 h in the absence of light and at room temperature was carried out. The total hexose content of the extract was determined according to the method of Hanssen and Moller (1975) in which the quantity of C6 sugars is determined by colorimetric determination. A solution of anthrone solubilised in sulfuric acid will specifically react with the sugars, turning from yellow to green. The absorbance of the complex is read on the spectrophotometer at 625 nm. The sugar content is determined using a standard glucose curve.

The results presented in FIG. 2 show a decrease in the glucose concentration in the culture medium at t=12 h, only in the culture medium containing Dunaliella salina, meaning that the microalgae consumed and metabolised glucose.

Amino acids are an important nitrogen source for microalgae growing in natural environments. Amino acids have been shown to form a significant proportion of the dissolved nitrogen in marine waters and could provide an additional source of nitrogen for the growth of marine microalgae. Some studies suggest that maximum amino acid capture rates occur under conditions of darkness in waters depleted in dissolved inorganic nitrogen.

The ability of Dunaliella salina to grow under heterotrophic conditions with glucose as a carbonaceous nutrient source and a yeast extract very rich in nitrogenous molecules such as amino acids as a nitrogen source was evaluated. The culture in this case takes place in the dark, which is conducive to the metabolism of the amino acids present in the culture medium.

In order to accurately determine the amino acids contained in the culture medium at room temperature at t=0 and t=12 h in the absence of light, the samples were analysed by HPLC-DAD. The culture media containing the microalgae Dunaliella salina at t=0 and t=12 h were centrifuged to remove the microalgae, in order to determine only the amino acids present in the culture medium. The samples were separated by an Uptisphere Strategy C18-2 5 μm (250×4.6 mm) US5C182-250/046 (Interchim reference: UE2.6AQ-100/046) on an Agilent 1260 HPLC system (Agilent Technologies, CA, USA). The flow rate is 1 ml/min. The mobile phase consists of 0.1% phosphoric acid (H3PO4): solution (A) and acetonitrile: solution (B). Table 1 below shows the gradient programme for elution.

TABLE 1

| Time (min) | % (H3PO4 0.1%) | % (ACN) |
|---|---|---|
| 0 | 87 | 13 |
| 20 | 54 | 46 |
| 22 | 54 | 46 |
| 27 | 60 | 40 |
| 30 | 60 | 40 |
| 35 | 5 | 95 |
| 37 | 87 | 13 |

The temperature of the column was maintained at 25° C. The injection volume was 5 μL and the detection wavelength was set at 254 nm using a UV detector. The amino acid standards were purchased from Sigma-Aldrich. The standards and the sample before injection are derived with phenylisothiocyanate (PITC). Amino acid identification was performed by comparing the retention times and UV spectral peaks of the sample to standard amino acids.

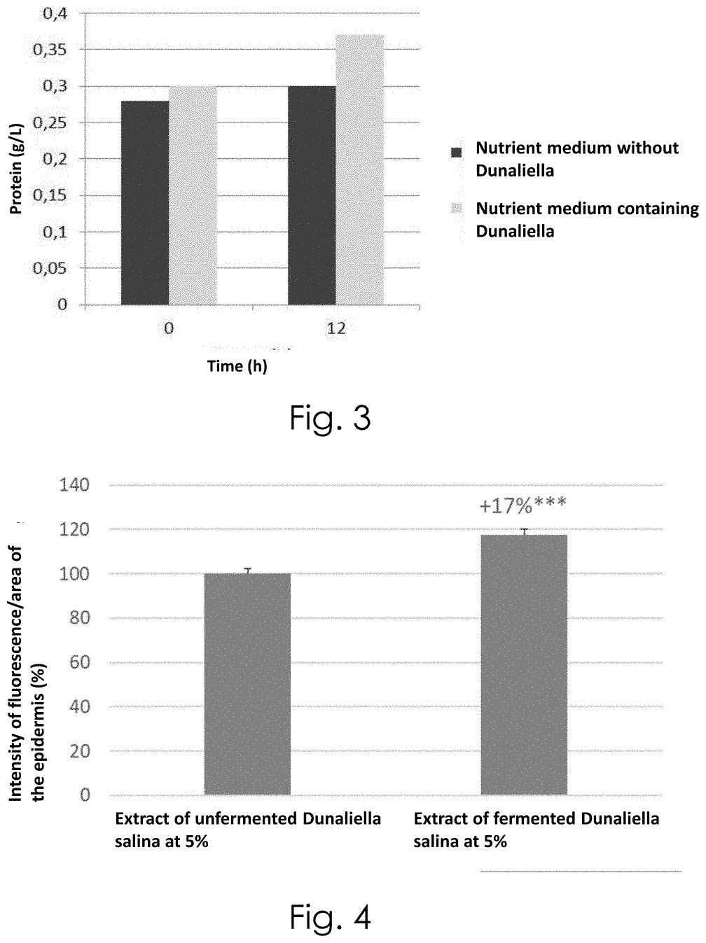

The concentration of certain amino acids decreased significantly under the condition of a culture medium containing Dunaliella salina after t=12 h of fermentation compared to the time t=0, in particular for serine, asparagine, aspartic acid, alanine, arginine, glutamic acid. These different amino acids were consumed by the microalgae during fermentation, and were used in part for the synthesis of the proteins of the growing microalgae. This is shown in FIG. 3, where the protein content of a “microalgae plus nutrient medium” extract is higher after 12 hours of fermentation.

### Example 5: Evaluation of the Effect of the Aqueous Extract of Fermented

Dunaliella salina, prepared according to Example 1, on the barrier function and movement of water on skin ex vivo

The purpose of this study is to show the effect of the fermented Dunaliella salina aqueous extract, prepared according to Example 1, without the addition of preservatives, compared to the unfermented Dunaliella salina aqueous extract, prepared according to Example 3, on barrier function and water movement by evaluating claudin-1.

The skin is the interface between the body and the external environment. It aims to protect the body from external aggressions but also to fight against dehydration by limiting the diffusion of water. The maintenance of hydrous homeostasis is ensured by various elements including the tight junctions of the granular layer. These junctions, consisting in part of the transmembrane protein claudin-1, prevent passive diffusion of solutes and water through the intercellular space. Finally, it has been observed that claudin-1 expression decreases with skin aging and claudin-1 deficiency leads to the appearance of wrinkles (Furuse et al., “Claudin-based tight junctions are crucial for the mammalian epidermal barrier: a lesson from claudin-1-deficient mice”, J Cell Biol. 156(6):1099-111, 2002; Jin S. P. et al, “Changes in tight junction protein expression in intrinsic aging and photoaging in human skin in vivo”, J Dermatol Sci. 97-113/2016

Protocol:

Human skin biopsies of 6 mm diameter were maintained ex vivo in a specific culture medium (DMEM at 1 g/L, HAMF12, foetal calf serum and antibiotics). The biopsies were treated twice a day for 48 hours with either a fermented Dunaliella salina extract solution, prepared according to Example 1, or an unfermented Dunaliella salina extract solution, prepared according to Example 3, both diluted in PBS to a final concentration of 5% vol/vol.

For the immunostaining of claudin-1, the tissues are fixed and embedded in paraffin. The skin biopsies thus included are cut and the sections are dewaxed and rehydrated. Then, an unmasking protocol is carried out before the application of a specific anti-claudin-1 antibody (Abcam, ab15098, rabbit polyclonal), followed by a suitable secondary antibody coupled with a fluorescent dye. After being mounted in a particular medium, the slides are observed with an epifluorescence microscope (Zeiss Axiovert 200M microscope).

Results:

As shown in FIG. 4, treatment with the fermented Dunaliella salina extract solution diluted 5% in PBS showed a significant increase (***) of 17% in claudin-1 expression compared to unfermented Dunaliella salina extract solution diluted 5% in PBS, in ex vivo skin biopsies.

Conclusion:

After application of a solution of fermented Dunaliella salina extract diluted to 5%, we observe an increase in the expression of claudin-1. Claudin-1 is associated with the barrier function and movement of water in the skin.

### Example 6: Formulations

In the following formulas, the fermented Dunaliella salina extract used was obtained according to Example 1 and 8% Dead Sea salt was added as a preservative. It is called: Fermentation Mineral Juice (FERMENT MINERAL BROTH).

### Example 6.1: Moisturising Day Cream

Phase A

| Water | qs |
|---|---|
| Glycerine | 3 |
| Aloe Vera | 0.1 |
| Sodium hyaluronate | 0.2 |
| Ascorbyl glucoside | 3 |

Phase B

| | |
|---|---|
| OLIVEM 1000 ® = Cetearyl Olivate, Sorbitan Olivate | 5 |
| OLIWAX ® LC = Cetyl Palmitate, Sorbitan Palmitate, | 1 |
| Sorbitan Olivate | |
| Argan oil | 1 |
| Phytosqualane | 4 |
| Vitamin E = Tocopheryl Acetate | |

Phase C

| | |
|---|---|
| Preservative | 1 |

Phase D

| | |
|---|---|
| Fragrance | 0.6 |
| Fermentation Mineral Juice (FERMENT MINERAL BROTH) | 1 |

### Example 6.2: Facial Serum

Phase A

| Water | qs |
|---|---|
| Glycerine | 3 |
| Aloe Vera | 0.1 |
| Sodium hyaluronate | 0.1 |
| Ascorbyl glucoside | 5 |

Phase B

| | |
|---|---|
| SIMULGEL ® INS 100 = Hydroxyethyl Acrylate/Sodium | 60 |
| Acryloyldimethyl Taurate Copolymer (and) Isohexadecane (and) | |
| Polysorbate | |
| Grape seed oil | 1 |
| Phytosqualane | 4 |
| Vitamin E = Tocopheryl Acetate | |

Phase C

| | |
|---|---|
| Fermentation Mineral Juice (FERMENT MINERAL BROTH) | 2 |
| Preservative | 1 |

Phase D

| | |
|---|---|
| Fragrance | 0.6 |

### Example 6.3: Facial Essence

Phase A

| Water | qs |
|---|---|
| Fermentation Mineral Juice (FERMENT MINERAL BROTH) | 30 |
| Glycerine | 3 |
| FUCOGEL ® = Biosaccharide Gum-1 | 1 |
| Fragrance | 0.3 |
| Red algae extract | 0.01 |

### Example 6.4: Night Mask

Phase A

| | |
|---|---|
| Water | qs |
| Cactus extract | |
| Sodium lactate | |
| Aloe vera | |
| Allantoin | |

Phase B

| | |
|---|---|
| TEGOCARE ® 450 = Polyglyceryl-3 Methylglucose Distearate | 1 |
| DERMOWAX ® SB = Stearyl behenate | 0.2 |
| INUTEC ® SL1 = Glycerin, Inulin Lauryl Carbamate | 0.2 |
| Isononyl isononanoate | 0.5 |

Phase C

| | |
|---|---|
| Preservative | 0.5 |
| TWEEN ® 20 = Polysorbate 20 | 0.1 |
| Lavender essential oil | 0.5 |

Phase D

| | |
|---|---|
| Fermentation Mineral Juice (FERMENT MINERAL BROTH) | 5 |

### Example 6.5: Dead Sea Shower Gel

Phase A

| Water | qs |
|---|---|
| Glycerine | 10 |
| POLYQUATERNIUM-10 | 0.3 |
| Citric acid | 0.5 |
| JAGUAR ® C17 = Guar Hydroxypropyltrimonium Chloride | 0.3 |

Phase B

| | |
|---|---|
| MIRACARE ® SLB 365W = Water (and) Sodium trideceth | 28 |
| sulphate (and) Cocamide MEA (and) Sodium Lauroamphoacetate | |
| (and) Sodium Chloride | |

Phase C

| | |
|---|---|
| Jojoba oil | 3 |
| Fragrance | 1 |

Phase D

| | |
|---|---|
| Preservative EUXYL ® 9010 = Phenoxyethanol (and) | 1 |
| Ethylhexylglycerine | |

Phase E

| | |
|---|---|
| Fermentation Mineral Juice (FERMENT MINERAL BROTH) | 1 |

### Example 6.6: Dead Sea Shampoo

Phase A

| Water | qs |
|---|---|
| POLYQUATERNIUM-10 | 0.2 |
| Panthenol | 0.15 |
| ELFACOS ® GT 282S = Ceteareth-60 Myristyl Glycol | 0.7 |
| Citric acid | 0.25 |

Phase B

| | |
|---|---|
| Water | 1 |
| Dead Sea mud | 0.5 |
| Fermentation Mineral Juice (FERMENT MINERAL BROTH) | 1 |

Phase C

| | |
|---|---|
| Sodium laureth sulphate | 8.5 |
| MIRANOL ® C2M = Disodium Cocoamphodiacetate | 12 |
| REWODERM ® S1333 = Disodium Ricinoleamido MEA- | 2.1 |
| Sulfosuccinate | |

Phase D

| | |
|---|---|
| EUXYL ® 9010 = Phenoxyethanol (and) Ethylhexylglycerine | 1.1 |
| Tocopheryl acetate | 0.05 |
| Argan oil | 0.1 |
| Fragrance | 0.8 |

### Example 6.7: Dead Sea Hair Conditioner

Phase A

| | |
|---|---|
| Water | qs |
| Glycerine | 1 |
| Niacinamide | 0.2 |
| Behentrimonium chloride | 2 |
| POLYQUATERNIUM-10 | 0.3 |
| DOW CORNING ® 939 emulsion = Amodimethicone (and) | 1 |
| cetrimonium chloride (and) TRIDECETH-12 ® | |

Phase B

| | |
|---|---|
| Isopropyl myristate | 2 |
| Argan oil | 1 |
| Cetearyl alcohol | 5 |
| DOW CORNING ® 200 fluid = Dimethicone | 1.5 |

Phase C

| | |
|---|---|
| Water | 1 |
| Dead Sea Mud | 1 |
| Fermentation Mineral Juice (FERMENT MINERAL BROTH) | 1 |

Phase D

| | |
|---|---|
| EUXYL ® 9010 = Phenoxyethanol (and) Ethylhexylglycerine | 1.1 |
| Tocopheryl acetate | 0.05 |
| Fragrance | 1 |

Phase E

| | |
|---|---|
| Green tea extract | 0.5 |
| Chamomile extract | 0.5 |
| Pomegranate extract | 0.5 |
| SILK TEIN ® NPNF = hydrolysed silk | 0.5 |

### Example 6.8: Dead Sea Hair Mask

Phase A

| Water | qs |
|---|---|
| Glycerine | 3 |
| Lactic acid | 1 |
| Panthenol | 1 |
| Behentrimonium chloride | 1.5 |
| DOW CORNING ® 939 emulsion = Amodimethicone (and) | 4 |
| cetrimonium chloride (and) TRIDECETH-12 ® | |
| POLYQUATERNIUM-10 | 2 |

Phase B

| | |
|---|---|
| Cyclopentasiloxane SF 1202 | 2 |
| ABIL QUAT ® 3474 Quaternium-80 | 2 |
| MYRITOL ® 318 = Caprylic/Capric Triglyceride | 0.5 |
| Argan oil | 0.1 |
| Cetearyl alcohol | 9.5 |
| Glyceryl stearate | 1 |

Phase C

| | |
|---|---|
| EUXYL ® 9010 = Phenoxyethanol (and) Ethylhexylglycerine | 1.1 |

Phase D

| | |
|---|---|
| Dead Sea Mud | 1 |
| Fermentation Mineral Juice (FERMENT MINERAL BROTH) | 5 |

Phase E

| | |
|---|---|
| Fragrance | 1 |
| Tocopheryl acetate | 0.5 |

Phase F

| | |
|---|---|
| Silk protein G | 0.1 |
| FISION KERAVEG ® 18 = Water (and) Wheat Amino Acids (and) | 0.7 |
| Soy Amino Acids (and) Arginine HCl (and) Serine (and) Threonine | |
| Colouring agent | 0.1 |

1. A method for obtaining an aqueous extract of a Dunaliella salina microalgae, wherein said microalgae is cultured in the absence of light in a culture medium comprising a yeast extract, sugar and salt, the method comprising:
a) solubilising a yeast extract in water;
b) adding glucose to the mixture obtained in a);
c) adding Dead Sea salt;
d) after total solubilisation of the salt in the mixture obtained in b), the culture medium thus obtained has a pH of between 5 and 8, and the microalgae Dunaliella salina is added thereto;
e) moderately stirring the mixture obtained in d), in darkness and at room temperature, for a period of at least 12 hours, in order to allow the fermentation of the microalgae Dunaliella salina;
f) grinding and then filtering the fermented Dunaliella salina mixture obtained in e) in order to separate the soluble and insoluble materials;
g) recovering a soluble aqueous raw extract to which Dead Sea salt or a preservative such as sodium benzoate is added;
h) carrying out sterilising filtration with a porosity threshold of less than or equal to 0.2 μm; and
i) obtaining a fermented aqueous extract of Dunaliella salina in which the pH is between 3.5 and 4.5.

2. The method according to claim 1, wherein in a) a yeast extract in powder form is solubilised in distilled water in a ratio of yeast extract/water of 0.1 to 2% w/w.

3. The method according to claim 1, wherein in b) glucose is added in a concentration of between 0.1 and 4% by weight of the total weight of the mixture.

4. The method according to any one of claim 1, wherein d) and i) comprise adjusting the pH by adding a solution of hydrochloric acid (HCl) or sodium hydroxide (NaOH).

5. The method according to claim 4, wherein in i) the pH is between 3.5 and 4.0.

6. An aqueous extract of Dunaliella salina obtained by the method according to claim 1, wherein the aqueous extract of Dunaliella salina comprises, by weight of the total weight of the extract, from 15 to 25 g/kg dry weight, 0.1 to 2 g/kg protein fragments, 0.3 to 3 g/kg sugars, 0.5 to 3 g/kg amino acids and 20 to 150 mg/kg phenolic compounds.

7. A composition comprising, as an active anti-aging agent, an effective amount of an aqueous extract of Dunaliella salina according to claim 6, and a physiologically acceptable medium.

8. The composition according to claim 7, wherein the effective amount of the aqueous extract of Dunaliella salina is at a concentration of 0.1 to 10% by weight of to the total weight of the composition.

9-10. (canceled)

11. The method according to claim 2, wherein in step a) a yeast extract in powder form is solubilised in distilled water in a ratio of yeast extract/water of 0.2%.

12. The method according to claim 3, wherein in step b) glucose is added in a concentration of 2% by weight of the total weight of the mixture.

13. The method according to claim 4, wherein in i) the pH is 4.0.

14. The composition according to claim 8, wherein the effective amount of the aqueous extract of Dunaliella salina is at a concentration of 0.5% to 5% by weight of to the total weight of the composition.

15. A method of treating skin, scalp, or skin appendage comprising:
providing a composition comprising, as an active anti-aging agent, an effective amount of an aqueous extract of Dunaliella salina in a physiologically acceptable medium;
wherein the aqueous extract of Dunaliella salina comprises, by weight of the total weight of the extract, from 15 to 25 g/kg dry weight, 0.1 to 2 g/kg protein fragments, 0.3 to 3 g/kg sugars, 0.5 to 3 g/kg amino acids and 20 to 150 mg/kg phenolic compounds;
applying the composition topically to skin, scalp, and/or skin appendage in need thereof.

16. The method according to claim 15, wherein the composition improves the appearance of the skin, scalp, and/or skin appendage, thereby combating the signs of skin aging or improving the hydration of the skin and strengthening the barrier function.

17. The method according to claim 15, wherein the composition is a cosmetic composition.

18. The method according to claim 15, wherein the composition is a hair treatment or shampoo.

19. The method according to claim 15, wherein the composition is a cream, lotion, milk, serum, ointment, gel, paste, foam, solid stick, or an aerosol.
